(19) United States
(12) Patent Application Publication
(10) Pub. No.: US 2022/0228986 A1
(43) Pub. Date: Jul. 21, 2022

(54) TAGGED PLANT MATERIAL AND METHOD FOR IDENTIFYING SAME

(71) Applicant: DOTZ NANO LTD., Petach-Tikva (IL)

(72) Inventors: Yonit BOGUSLAVSKY, Petach-Tikva (IL); Yoni ENGEL, Ra'anana (IL); Inna SASSON, Givataim (IL); Michael Shtein, Kfar Daniel (IL); Tanya Dyshel, Rehovot (IL)

(21) Appl. No.: 17/608,521

(22) PCT Filed: Apr. 27, 2020
(86) PCT No.: PCT/IL2020/050471

§ 371 Date: Nov. 3, 2021

(30) Foreign Application Priority Data
May 5, 2019 (IL) 266444

(51) Int. Cl. G01N 21/64; C09K 11/65; G01N 33/00

(52) U.S. Cl. CPC G01N 21/6428; G01N 2021/6439; G01N 33/0098; C09K 11/65

(57) ABSTRACT

A method for tagging a plant growth comprises incorporating one or more fluorescent carbon dots into said plant growth.

### FIELD OF THE INVENTION

The present invention relates to a device, systems and methods to semi-forensically and forensically detect security markers embedded inside plants of value. Specifically, the invention relates to a method which makes use of a combination of fluorescent markers and dedicated detectors adapted to selectively excite and detect emission of a preconfigured pattern of photoluminescence sources and their use for authenticating Cannabis or other plans of interests.

### BACKGROUND OF THE INVENTION

The Cannabis industry is rapidly growing, with more and more entities being licensed in various countries to grow, process, and distribute cannabis. In parallel, government regulations in various countries and states require manufacturers and distributors to take active action (among others), to:
Prevent distribution of Cannabis related products to minors;
Prevent the diversion of Cannabis related products from states where it is legal under state law in some form to other states;
Prove, with high level of certainty that their products and raw materials originate from legal sources.

In addition, some governments also require that Cannabis related products be taxed at a certain rate different than that of neighboring states. In light of the above—Counterfeiting, diversion, and unauthorized distribution and sale of various cannabis related products throughout the entire supply chain, such as fractions extracted from the plant or products containing plant material and/or fractions originating from the plant, have emerged as substantial challenges for the cannabis industry.

Throughout the supply chain, cannabis growers, processors, distributors, and retailers currently use radio-identification tags (RFID) which require physical attachment to the package containing the cannabis or to the stem of the plant. Ideally, this system provides regulators with a tool to monitor the cannabis chain of custody, however in practice its dependence on a physical attachment of a clearly visible tag exposes it to removal, destruction or counterfeiting. Furthermore, diversion of cannabis products to illegal markets may occur intentionally by producers and distributors that are trusted to be the ones placing the physical tag on the plant. Such rogue growers/distributors will simply avoid placing the tag on the portion of products they wish to divert to illegal markets. Moreover, in cases where the cannabis product is processed (e.g. extracted to produce cannabis oil or cannabis water extract) the taggant is removed and no indication of the source is available.

Other solutions, which include spraying of a molecular marker onto the surface of the plant and forensically detecting it at a later stage, can be removed by washing and are susceptible to destruction and degradation by exposure to sun light, UV-light, or heating which are often encountered during the cannabis supply chain.

It would therefore be highly desirable to be able to provide a method for tagging the cannabis plant with a robust marker that cannot be easily removed or destroyed throughout the supply chain, even if the plant is processed.

Furthermore, it would be highly desirable to be able to provide a taggant material which is detectable either externally in a non-destructive way, or destructively by extraction of the taggant material from the plant, allowing for multiple ways to track and protect the cannabis plant along the supply chain.

It is a purpose of the present invention to provide such tagging materials, which fulfill the above-mentioned requirements of stability and robustness.

It is another purpose of the invention to provide tagging materials, which can be effectively incorporated into plants without adversely affecting their growth or properties, which are not toxic to the plant or to the user of its product, and which are sufficiently inexpensive that they can be used on a large scale.

It is yet another object of the present invention to provide a marker-detecting device, which operates in combination with markers that are suitable to be used in plants.

Other objects and advantages of the invention will become apparent as the description proceeds.

### SUMMARY OF THE INVENTION

The invention relates to a method for tagging a plant growth, comprising incorporating one or more fluorescent carbon dots into said plant growth. In one embodiment the incorporation is performed by irrigation. In another embodiment the incorporation is performed by wetting the plant foliage.

In one embodiment, the fluorescent carbon dots comprise two or more carbon dots having differently detectable maximum excitation/emission wavelengths.

The plant growth can be of any type, and in one exemplary embodiment the plant growth is selected from cannabis and Saffron.

The invention is also directed to a method for identifying and/or verifying the origin of plant material that was tagged with one or more carbon dots, comprising subjecting said plant material to excitation at a range of wavelengths, recording the emission obtained thereby, and comparing said emission to reference values. In one embodiment the plant material is a live plant. In another embodiment the plant material is a dried plant material. In a further embodiment the plant material is a plant extract.

Also encompassed by the invention is plant material tagged with one or more fluorescent carbon dots.

### BRIEF DESCRIPTION OF THE FIGURES

In the drawings:

FIGS. 1 (A and B) show 2D-Fluorescence maps of an extract from reference plant (A) and of an extract from a plant irrigated with GQD 06-0332 (B);

FIG. 1C is the spectra overlay of fluorescent peak of extract from GQD 06-0332 treated plant and the reference plant;

FIGS. 2 (A and B) show 2D-Fluorescence maps of an extract from reference plant (A), and of an extract from a plant irrigated with GQD 06-0336 (B);

FIG. 2C shows the Photoluminescence (PL) spectra of extract from GQD 06-0336 treated plant and of extract from the reference plant;

FIGS. 3 (A and B) are 2D-Fluorescence maps of an extract from reference plant (A) and of an extract from a plant irrigated with GQD 06-0340 (B);

FIG. 3C shows the PL spectra of an extract from a GQD 06-0340 treated plant, and of an extract from the reference plant;

FIG. 4 is a grayscale image (taken under UV lamp) of a leaf cut from the reference plant (1), a leaf from a GQD 06-0332 irrigated plant (2), and a leaf from a GQD 06-0336 irrigated plant (3);

FIG. 5 (A and B) show 2D-Fluorescence maps of extracts from a reference plant (A) and extract from a plant irrigated with GQD 06-0332 (B);

FIG. 5C is the spectra overlay of a fluorescent peak of extract from GQD 06-0332 treated plant, and from the reference plant;

FIGS. 6 (A and B) show 2D-Fluorescence maps of an extract from a reference plant (A), and of an extract from a plant irrigated with GQD 06-0336 (B);

FIG. 6C is the PL spectra of an extract from a GQD 06-0336 treated plant, and of an extract from a reference plant;

FIGS. 7 (A and B) are 2D-Fluorescence maps of an extract from reference plant (A), and an extract from a plant irrigated with GQD 06-0340 (B);

FIG. 7C is the PL spectra of an extract from a GQD 06-0340 treated plant and from a reference plant;

FIG. 8 shows a system comprising a device with a detector configured to detect photonic emission from a substrate;

FIG. 9 schematically illustrates the setup of device 100 of FIG. 8;

FIG. 10 illustrates the different levels of detection that can be achieved by inspecting a tagged plant;

FIG. 11 illustrates the stability of GQD 06-0336 in dried plants during 5 months of storage. Numerals 1, 2, 3 relate to the number of each tagged plant (triplicates were used); r-1, r-2, r-3 are the designation of reference plants (triplicates were used);

FIG. 12 illustrates the stability of GQD 06-033240 in dried plants during 5 months of storage. Numerals 1, 2, 3 relate to the number of each tagged plant (triplicates were used); r-1, r-2, r-3 are the designations of reference plants (triplicates were used); and

FIG. 13 shows how the detection of carbon dots in leaves is performed in one embodiment of the invention.

### DETAILED DESCRIPTION OF EMBODIMENTS

Carbon dots (CDs) are aggregated non-conjugated organic polymer structures. The bottom up synthesis of carbon dots permits to achieve molecular and supramolecular carbon structures with enhanced fluorescent properties and tunable spectral and chemical properties. Furthermore, while being extremely polar and water soluble, CDs are considerably more resistant to temperature, pH changes, hydrolysis, light, bio-degradation, oxidation, chemical attack, or adsorption to rock structures making them particularly useful for tagging applications in the cannabis plant through soil or foliage uptake.

A side benefit of the use of carbon dots with plants is that they promote plant growth, including root elongation, stem elongation, and biomass. Accordingly, not only carbon dots do not present disadvantages in the course of plant growing, but they are actually beneficial in many cases, as compared to a variety of tagging materials, which may actually be deleterious to plant growth.

As used herein, the term “fluorescent carbon-based materials” relates to carbon materials having fluorescence properties (for the sake of brevity, the term “fluorescence” are used herein also to refer interchangeably to photoluminescence (PL)). In the context of this description, the “fluorescent carbon-based materials” encompass carbon molecules, carbon-based oligomers and polymer/co-polymer structures, carbon dots (CDs), photoluminescent carbon nanostructures (PCNs) such as graphene quantum dots (fluorescent carbon-based materials), graphene oxide quantum dots, carbon nanotube quantum dots or a combination of one or more of said materials. Specifically, the fluorescent carbon-based materials may originate from any organic carbon source which is non-toxic or otherwise not deleterious to the intended use. The carbon nanotube quantum dots can be single wall nanotube (SWNT), or multi-wall nanotube (MWNT), or a combination thereof.

Apparatus useful in connection with the present invention comprises hyperspectral/multispectral (also referred to herein as “imaging system”, “fluorimeter”, and the like) imaging systems, comprising: an illumination module configured to illuminate a subject; an optical acquisition module adapted to acquire photons emitted from the subject; a display a central processing module (CPM); a display in communication with the CPM; and a memory in communication with the CPM having thereon a processor-readable medium with a set of executable instructions configured to cause the CPM to: in response to activation by a user, illuminate the subject; detect photons emitted from the subject; and display a pattern represented by the emitted photons. Such a practice is known per se and, therefore, is not described herein in detail for the sake of brevity.

As explained above, provided herein are embodiments of systems, devices and methods for detecting a preconfigured spectral pattern and uses thereof. Specifically, the disclosure relates to a device adapted to selectively excite and detect the emission of a preconfigured spectrum (e.g., color) of fluorescent carbon-based materials, and their use for authenticating plants of value. The plants of value that can be tagged, identified and authenticated using the systems, devices, and methods described herein can be, for example, cannabis plant, cannabis oil, cannabis water extracts, Saffron or other plants, vegetables, or fruit of value where source identifying is important to ensure consumers, producers, distributors and government of the origin of the items thus tagged.

Detection of the tagged plants can be effected by a variety of equipment, for instance, that schematically shown in FIG. 8, which consists of a system 10, comprising a device 100 with a detector configured to detect photonic emission from a substrate. The device of FIG. 8 is provided with a detector, a power source and a plurality of excitation sources for emitting electromagnetic radiation (EMR), as further discussed herein below. The device can be configured to be handheld, and in the embodiment shown in the figure, it is powered by a smart phone 200, via cable 150, connected, in this particular example, via USB port 250. Of course, alternatively the device can be connected wirelessly, e.g., via Bluetooth. Device 100 can then recognize a specific substrate, schematically indicated in the figure by 300. The plurality of excitation sources for emitting electromagnetic radiation (EMR) in the device and systems described herein, can be adapted to emit EMR with narrow full width at half maximum (FWHM). The electromagnetic radiation source can be a light emitting diode (LED) adapted to provide light at a discrete wavelength, a LASER source (e.g., a laser diode or diodes), or a light source coupled to appropriate optical filter. As indicated, there can be more than one LED thus providing simultaneous excitation at various wavelengths, as there can be more than a single LASER source or light source with optical filters that limit the wavelength spectrum exciting the fluorescent carbon-based materials, for example, carbon polymer dots (CPDs).

The EMR emitting sources can be, for example, light emitting diodes (LEDs) configured to emit light at a very narrow wavelength without generating heat. In an embodiment, the LEDs can be configured to have a central wavelength (CWL) that coincides with the peak excitation wavelength of fluorescent carbon-based materials forming the pattern or configured to provide a particular color combination (spectra), while the full width at half maximum (FWHM) can be configured to be narrow enough so as not to substantially overlap with the excitation wavelength of other fluorescent carbon-based materials forming the pattern and/or the desired spectra. For example, FWHM of the EMR sources can be between 10 nm and 20 nm, while the FWHM of the photoluminescence sources (PCNs) can be configured to be between 30-40 nm.

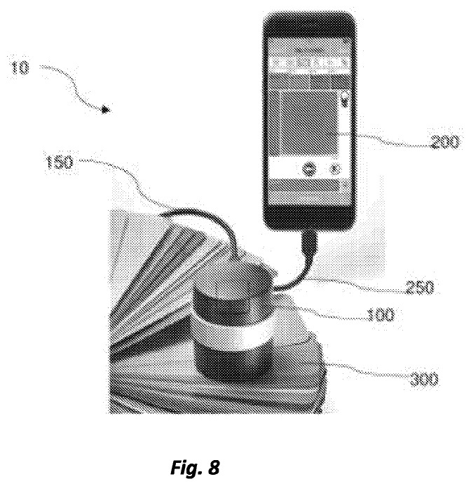

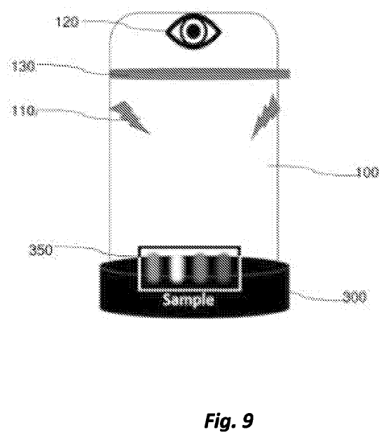

FIG. 9 schematically illustrates the setup of device 100. In the figure, 120 is an actuating button, 130 is an optical filter that can be switched between different filters, 110 indicates one or more excitation sources, 300 is the base of device 100, and 350 schematically illustrates samples being analyzed by device 100.

The detector can be used to detect only color emitted from fluorescent carbon sources embedded homogenously into a plant of interest. Notably, since the fluorescent carbon-based materials used in the compositions and methods provided herein are stable at high temperatures, high pressure and shear force, show relative inertness to changing chemical environments and are usually more photostable than organic dyes which may perform similarly in terms of initial Quantum Yield (QY), they improve the durability of the marking. This, in combination with their high QY enables their loading into plants of value in ultra-low levels (e.g., about 10 ppm in various systems) while retaining significant signal.

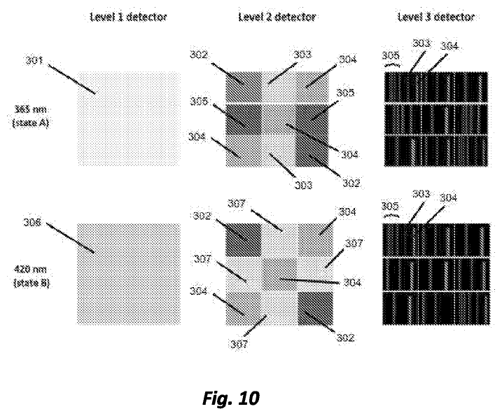

FIG. 10 illustrates the different levels of detection that can be achieved by inspecting a tagged plant. Looking at the 365 nm range, level 1 detector level 2 detector and level 3 detector provide very different levels of detection. The different levels represent different spatial and spectral resolutions. For example, level 2 has a greater spatial resolution than level 1, and level 3 has a greater spectral resolution. Moreover, FIG. 10 only shows two states (A and B), but of course other states can be used, at different wavelength, and a scanning procedure may involve a plurality of such states. The colors in this figure are identified as follows: 301—cyan; 302—dark green; 303—blue; 304—orange; 305—purple; 306—light green; 307—turquoise. As can be easily seen in the figure, a variety of identifying patterns can be generated such that when read in a specific sequence using different level detectors, a clear identification of a tagged product can be achieved.

The term “system” as used herein should be taken to include any collection of systems or sub-systems that individually or jointly execute a set, or multiple sets, of instructions to perform one or more functions. Also, the term “system” refers to a logical assembly arrangement of multiple devices, and is not restricted to an arrangement wherein all of the component devices are in the same housing. Accordingly and in an embodiment, provided herein is a hyperspectral/multispectral imaging system, comprising: an illumination module configured to illuminate a subject using a plurality of discrete wavelengths in a predetermined sequence; an optical acquisition module adapted to acquire photons emitted from the subject; a display; a central processing module (CPM); a display in communication with the CPM; and a memory in communication with the CPM having thereon a processor-readable medium with a set of executable instructions configured to: in response to activation by a user, illuminate the subject; detect photons emitted from the subject; and display a pattern represented by the emitted photons.

The system and devices disclosed herein can be adapted to perform hyperspectral and/or multispectral imaging, referring to methods and devices for acquiring hyperspectral and/or multispectral data sets or data-cubes, which typically comprise images where continuously sampled, finely resolved spectral information is provided at each pixel (see e.g., FIG. 10). Additionally, or alternatively, the imaging device 100 of FIG. 8 may be a multi-spectral imaging device having a plurality of sensors 120 (as shown in FIG. 9) for collecting spectra (and thus intensity) data in a plurality of different wavelengths, for example 3 to 10 in number (see e.g., FIG. 10), from the predetermined fluorescent carbon-based pattern in the AOI. For example, multi-spectral imaging device 100 may be configured to collect spectra data in 4 or more different wavelengths, or 6 or more different wavelengths.

The term ‘module’, as used herein, means, but is not limited to, a software and/or hardware component, such as a Field Programmable Gate-Array (FPGA) or Application-Specific Integrated Circuit (ASIC), which performs certain tasks. A module may advantageously be configured to reside on the addressable storage medium and configured to execute on one or more processors. Thus, a module may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. The functionality provided for in the components and modules may be combined into fewer components and modules or further separated into additional components and modules.

In an embodiment, the illumination module used in the devices and systems described herein, implemented in the methods described, can comprise a plurality of electromagnetic radiation emitters configured to emit radiation at a predetermined wavelength range. For example, the plurality (e.g., one EMR emitter per fluorescent carbon-based materials. EMR emitters can be LEDs with discrete central wavelength (CWL), for example, as illustrated in FIG. 10, between about 365 nm and about 425 nm (purple); between about 435 nm and about 480 nm (cyan); between about 365 nm and about 425 nm (purple); between about 495 nm and about 540 nm (green); and between about 600 nm and about 615 nm (purple), with FWHM in each of about 10 nm. Furthermore, the plurality of EMR emitters can be configured to illuminate the sample discretely (in other words, one at a time), in a preconfigured sequence. For example, illumination of a first AOI can have the sequence Green:Cyan:Orange; while illumination of a second AOI can have the sequence Purple:Cyan:Green. The response in each illumination sequence can then be recorded and form a part of the authentication procedure.

Further, wherein the optical acquisition module comprises a detection element that is a photodetector, a charge-coupled device (CCD), diode array, complimentary metal-oxide sensor device (CMOS), a focal plane array or a detection element comprising one or more of the foregoing. The optical acquisition module is configured generally to parse received image into multiple distinct classes based on which axes of the data-cube (portion or all the AOI) are sampled at a given instant. For example, using dispersive approaches to hyperspectral imaging, the optical acquisition module can instantaneously sample the pattern along the spectral axis (e.g., from about 190 nm to about 720 nm) and along one spatial axis (see e.g., top row, FIG. 10 level 2 detector), but the other spatial axis must be scanned in time to build up a full data-cube. In an embodiment, light that impinges on an entrance slit can be dispersed through a grating or a prism, and the dispersed light is imaged onto a two-dimensional detector array. By scanning the slit relative to the scene in a reciprocating (in other words, back-and-forth) manner, the full data cube is built up and displayed using the device/system display (see e.g., 200, FIG. 8).

Other methods can be used in other embodiments to provide the necessary quantitative measurements for detecting and authenticating the spectra (color) of fluorescent carbon-based materials described. For example, Infrared (IR) spectroscopy that is based on the interaction with chemical substances of infrared irradiation having a wavelength between 0.77 μm and 1000 μm. A segment of IR spectroscopy, referred to as near infrared (NIR) spectroscopies, uses radiation wavelengths between about 0.77 μm and about 2.5 μm. IR and NIR spectroscopies generally involves the absorption of radiation as it passes through a sample. The absorption frequencies can therefore provide information regarding the chemical and physical characteristics or the molecular structure of the irradiated substance and its composition.

Moreover, the CPM is in electronic communication with a library comprising the hyperspectral and/or multispectral patterns. The library can serve as reference for the pattern detected and can include expected emission spectra at each wavelength as a function of the spatial resolution used in the AOI. Accordingly, the set of executable instructions are further configured to cause the CPM to analyze the detected pattern, and to provide a comparison between the detected pattern and the pattern obtained from the library. The comparison can then be used to authenticate the plant of value as identical to the source, or as a fake if the degree of homology (in other words, identity) is below a certain threshold, for example, less than 95%, or less than 90%, or in circumstances where the plant of value has been subjected to intensive wear and tear, even less.

The output of the detection device can vary from providing the average value of chroma (the strength of the surface color), hue (the dominant wavelength mode) and lightness of the whole sample (AOI), as well as degree of saturation when discretely illuminated at each wavelength provided by the plurality of EMR emitters. These values can then be compared with stored values of an authentic sample and be used to verify the source of the sample. Moreover, when hyperspectral imaging is used in the detection, the actual emission spectral bands can be used.

Embodiments described herein may comprise or utilize a special purpose or general-purpose computer including computer hardware, such as, for example, one or more processors (e.g., central processing module, CPM) and system memory, as discussed in greater detail below. Embodiments described herein also include physical and other computer-readable media for carrying or storing computer-executable instructions and/or data structures. Such computer-readable media can be any available media that can be accessed by a general purpose or special purpose computer system. Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: computer storage media and transmission media.

In some embodiments, the fluorescent carbon-based materials can be nano-sized (of less than 10 nm in size) structures of carbon molecules (more than a single atom) having dimensionality that is anywhere from quasi-one dimension (e.g., quantum dot, nanoribbon, nanobelt), to three dimensional (e.g., multilayer graphene structures). Encompassed in these nano-sized structures, are graphene, graphdiyne, fullerene, nanocage, multilayer graphene dot, nanodiamond, nanotube, nanowire, nanohorn, or a carbon dots composition comprising one or more of the foregoing.

Detecting, which in another embodiment also includes quantifying emission spectra, can be done, by detecting luminescence of the measured pattern. Luminescence spectroscopy involves the measurement of photon emission from molecules. It can include photoluminescence such as fluorescence and phosphorescence, which are emissions from a substance resulting from its excitation by radiation absorption, as well as chemiluminescence, where the emission is induced by a chemical reaction. The emitted radiation is characteristic of the molecular structure, size and composition. Accordingly, manipulating the structure, size and composition of both the fluorescent carbon-based materials (interchangeable with CQDs), it is possible to fabricate an AOI having a detectable, repeatable pattern.

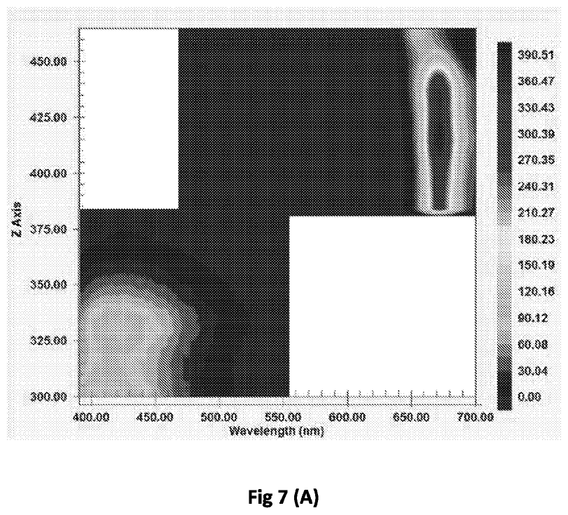

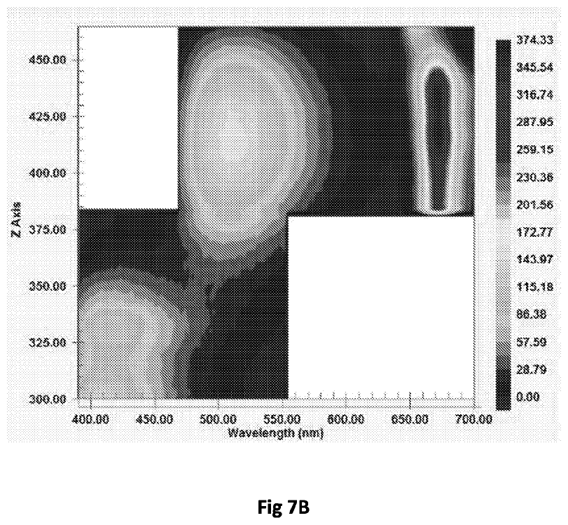

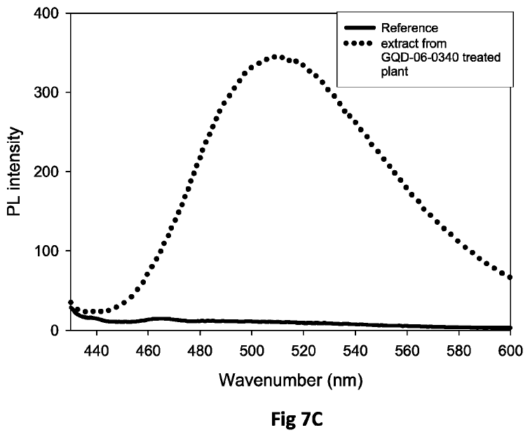

For example, by using a photodetector array (e.g., a PIN diode array) with different color filters and EMR sources based on the plurality of CODs used in the pattern, as well as provide an UV/VIS/NIR bandpass(es) color filter(s) operably coupled to a photodetector array. Alternatively, a (scanning) diffraction grating (in the case of hyperspectral imaging) coupled to a photodetector array can be used to determine the spectrum profile emitted from the plurality of CQDs. Detection can be quantified, yielding peak emission, half peak baseline, intensity and area under the curve (AUC), as well as ratios of the foregoing, as a function of excitation wavelength (see e.g., FIG. 7C); all which can be added to the linked library database at the device or system and used to compare with the test sample obtained by an end user or intermediate downstream (pipeline or supply chain).

As a point of clarification, the control over peak emission spectra of the photoluminescence sources is not necessarily solely a function of size, but of other factors as well, for example; the type of photoluminescence sources (e.g., fluorescent carbon-based materials, lanthanide nanorod, or MWCNT) the extent and location of surface defects in the fluorescent carbon-based materials when used, type and degree of substitution of various functional groups (e.g., carboxylate) as well as uniformity of size distribution and other factors. Accordingly, it is contemplated that fluorescent carbon-based materials having exactly the same overall average D3,2 particle size (e.g., ^{˜}5.0 nm), would nevertheless have peak emission spectra that is shifted between about 20 nm and about 80 nm.

One, all or some of electromagnetic radiation (EMR) sources can be incorporated in a handheld device housing having: a display; a processing module comprising a processor in communication with a linked library containing original pattern emission spectra at a specific wavelength, of the carbon quantum dot formed on the pattern in the AOI on the plant of value sought to be identified and/or authenticated; the processor further being in communication with: the plurality of electromagnetic radiation sources; a detector (e.g., a photodetector) configured to detect fluorescence, phosphorescence, chemiluminescence or their combination (and can further comprise additional optical color filters); the display; and a non-volatile memory having thereon a processor-readable medium with a set of executable instructions configured to: receive a reading from the detector; retrieve from the linked library a predetermined: emission spectra; and if the detected pattern emission spectra at a specific wavelength, retrieved from the detector correlates with the pattern emission spectra, at the predetermined specific wavelength, wavelength range or wavelength range segments that were retrieved from the linked library, authenticating plant of value using the display; else identifying the plant as non-authentic.

In an embodiment, the devices and systems described herein are being used in the methods disclosed. Accordingly, provided herein is a method of authenticating a plant of value having thereon an area of interest (AOI) adapted to emit photons at a predetermined spectra, comprising: using a hyperspectral/multispectral imaging system, detecting the spectra (color) of photons emitted by the plurality of CQDs embedded in the plant of value in response to exposure to a plurality of discrete EMR sourced emitting discrete wavelength at a predetermined sequence (wherein the discrete wavelength is between about 200 and 500 nm); comparing the detected spectra with a pattern associated with an authentic plant of value; and if the spectra detected is homologous to the pattern associated with the authentic plant of value, authenticating the plant; otherwise providing an indication that spectra detected are not homologous to the one associated with the authentic plant of value. In other words, comparing observed pattern and/or spectra vs. stored, data, whether remotely or locally. As indicated, the methods using the PCNs' patterns disclosed and claimed herein, implemented using the devices and systems described herein can further comprise comparing the detected emission pattern and/or spectra to a predetermined pattern and/or spectra corresponding to an authentic identity; and if the detected spectra correlates with the predetermined emission pattern and/or spectra, authenticating the article; else identifying the plant as non-authentic. The term “authentic” as used herein means that the pattern and/or spectra detected by the device has high correlation with the emission pattern and/or spectra obtained at the original source.

To clarify, in the description that follows, embodiments are described with reference to acts that are performed by one or more computing systems. These computing systems can be co-located or remote from each other and connected through various types of networks. The computing systems can be, for example, the handheld device disclosed, a backend management server with the pattern database library, and the like. If the computing systems are distributed (in other words not co-located or otherwise hardwired), the housing comprising the plurality of EMR sources (configured to emit EMR at discrete wavelength range of between NMT about 500 nm, or below full visible wavelength range), can further comprise a transceiver configured to initiate communication with remote computing systems. In other words, the EMR source is an actinic radiation source, configured to produce photochemical reaction in the pattern disposed on the substrate. So, for example, each EMR source can be configured to emit electromagnetic radiation at a wavelength between about 0.8 nm (laser equipped with power stabilizer) and about 450 nm, or between about 160 nm and about 300 nm, for example, between 190 nm and 250 nm.

If such steps are implemented in software, one or more processors of the associated computing system(s) (e.g., CPM) that performs the act direct the operation of the computing system in response to having executed computer-executable instructions. An example of such an operation involves the manipulation of data. The computer-executable instructions (and the manipulated data) may be stored in the memory of the computing system. Computing system may also contain communication channels that allow the computing system to communicate with other processors and sensors over, for example, service bus.

Embodiments described herein may comprise or utilize a special purpose or general-purpose computer including computer hardware, such as, for example, one or more processors (e.g., central processing module, CPM) and system memory, as discussed in greater detail below. Embodiments described herein also include physical and other computer-readable media for carrying or storing computer-executable instructions and/or data structures. Such computer-readable media can be any available media that can be accessed by a general purpose or special purpose computer system. Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: computer storage media and transmission media.

Computer storage media includes RAM, ROM, EEPROM, CD-ROM or other optical disk storage, magnetic disk storage or other magnetic storage devices, or any other medium which can be used to store desired program code means in the form of computer-executable instructions or data structures and which can be accessed by a general purpose or special purpose computer.

The term “module” is used herein to refer to software computer program code and/or any hardware or circuitry utilized to provide the functionality attributed to the module. Further, the term “module” or “component” can also refer to software objects or routines that execute on the computing system. The different components, modules, engines, and services described herein may be implemented as objects or processes that execute on the computing system (e.g., as separate threads).

Further, the CPM may be operably coupled to the various modules and components with appropriate circuitry. may also be used herein, the term(s) “operably coupled to”, “coupled to”, and/or “coupling” includes direct coupling between items and/or indirect coupling between items via an intervening item (e.g., an item includes, but is not limited to, a component, an element, a circuit, an engine, and/or a module) where, for indirect coupling, the intervening item does not modify the information of a signal but may adjust its current level, voltage level, and/or power level. As may further be used herein, inferred coupling (i.e., where one element is coupled to another element by inference) includes direct and indirect coupling between two items in the same manner as “coupled to”. As may even further be used herein, the term “operable to” or “operably coupled to” indicates that an item includes one or more of power connections, input(s), output(s), etc., to perform, when activated, one or more its corresponding functions and may further include inferred coupling to one or more other items. As may still further be used herein, the term “associated with”, includes direct and/or indirect coupling of separate items and/or one item being embedded within another item.

As may also be used herein, the terms “central processing module”, “module”, “processing circuit”, and/or “processing unit” may be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on hard coding of the circuitry and/or operational instructions. The processing module, processing circuit, and/or processing unit may have an associated memory and/or an integrated memory element, which may be a single memory device, a plurality of memory devices, and/or embedded circuitry of the processing module, module, processing circuit, and/or processing unit. Such a memory device may be a read-only memory, random access memory, volatile memory, non-volatile memory, static memory, dynamic memory, flash memory, cache memory, and/or any device that stores digital information. Note that if the processing module, module, processing circuit, and/or processing unit includes more than one processing device, the processing devices may be centrally located (e.g., directly coupled together via a wired and/or wireless bus structure) or may be distributed (e.g., cloud computing via indirect coupling via a local area network and/or a wide area network). Further note that if the processing module, module, processing circuit, and/or processing unit implements one or more of its functions via a state machine, analog circuitry, digital circuitry, and/or logic circuitry, the memory and/or memory element storing the corresponding operational instructions may be embedded within, or external to, the circuitry comprising the state machine, analog circuitry, digital circuitry, and/or logic circuitry. Still further note that, the memory element may store, and the processing module, module, processing circuit, and/or processing unit executes, hard coded and/or operational instructions corresponding to at least some of the steps and/or functions illustrated in one or more of the figures.

The terms “a”, “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the network(s) includes one or more network). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The term “system” shall also be taken to include any collection of systems or sub-systems that individually or jointly execute a set, or multiple sets, of instructions to perform one or more functions. Also, the term “system” refers to a logical assembly arrangement of multiple devices, and is not restricted to an arrangement wherein all of the component devices are in the same housing.

The method for authenticating or identifying a plant or an article of manufacture made therefrom includes in some embodiments applying a hyperspectral/multispectral imaging system as discussed above, detecting the pattern and/or spectra of photons emitted by the plant or plant article; comparing the detected pattern and/or spectra with a pattern and/or spectra associated with an authentic article of manufacture; and if the pattern and/or spectra detected is homologous to the pattern associated with the authentic article of manufacture, authenticating or otherwise identifying the article; otherwise providing an indication that the pattern detected is not homologous to the pattern associated with the authentic article of manufacture.

Although reference will be made herein primarily to cannabis, it should be understood that the same materials and procedures can be employed for the purpose of tagging, identifying and authenticating many other plant growths, and reference to cannabis only herein is made for the sake of brevity, cannabis been used as a representative plant growth.

### General Procedures

The following illustrates some of the many uses and procedures made possible by the methods and device of the invention.

### Procedure 1

Single or multiple fluorescent carbon dots are introduced into a live plant (e.g., cannabis) via uptake from soil or foliage following irrigation with water containing a carbon dots, completely solubilized or in emulsion form.

A multispectral device is used to resolve and authenticate a tagged live plant by examining the color generated by the emission of a single or multiple carbon dot present inside the plant through uptake. The measurement can be performed during growth of the plant in soil, after harvest, or following drying.

The sample is excited with light in different wavelengths (365 nm, 395 nm, 420 nm) and the emission and color response, resulting from the combination of the fluorescent carbon materials and the unique chemical environment inside the plant yields a different value in the color space for each wavelength. The resulting values are compared with a pre-stored reference sample to determine the authenticity/origin of the plant.

### Procedure 2

Single or multiple fluorescent carbon dots are introduced into a live plant via uptake from soil or foliage following irrigation with water containing a carbon dots, completely solubilized or in emulsion form. A hyperspectral device is used to resolve and authenticate a live or dried plant by examining the emission pattern generated by a single or multiple carbon dots present inside the plant through uptake. The measurement can be performed during growth of the plant in soil, after harvest, or following drying.

The sample is excited with light in different wavelengths (e.g. 365 nm, 395 nm, 420 nm) and the combination of a single or multiple fluorescent carbon materials and the unique chemical environment inside the plant yields a unique emission pattern for each excitation wavelength. The spectral line shape emitted upon excitation at each wavelength (since multiple illumination sources are used. The resultant spectral patterns are compared with a pre-stored reference sample to determine the authenticity/origin of the plant.

### Procedure 3

Single or multiple fluorescent carbon dots are introduced into a live plant via uptake from soil or foliage following irrigation with water containing a carbon dots, completely solubilized or in emulsion form. Fluorescent carbon dots present inside the live plant or extracted using a mixture of IPA/Water for 45 min at 100° C. Extraction can be performed during growth of the plant in soil, after harvest, or following drying.

The sample is filtered or used as is and is placed in a cuvette and measured using a fluorimeter and the resultant 3D excitation-emission map or otherwise emission at different excitation wavelength is recorded over the range between 200-1200 nm. The recorded spectra are compared with pre-stored reference data to determine the authenticity/origin of the plant.

### Procedure 4

Single or multiple fluorescent carbon dots are introduced into a live plant via uptake from soil or foliage following irrigation with water containing a carbon dots, completely solubilized or in emulsion form. Following harvest, oil is extracted from the plant using Ethanol or olive oil. The sample is evaporated and the oil is placed in a cuvette and measured using a fluorimeter and the resultant 3D excitation-emission map or otherwise emission at different excitation wavelength is recorded over the range between 200-1200 nm.

The recorded spectra are compared with pre-stored reference data to determine the authenticity/origin of the plant.

### Example 1

Three Capsicum plants were irrigated with three kinds of water-based Graphene Quantum Dots (GQDs), specifically GQD 06-0332, GQD 06-0336, and GQD 06-0340, available from STREM Chemicals, Inc., USA, (https://www.strem.com/catalog/v/96-7420/33/graphene_quantum_dots_in_water_gqds_mini_kit_liquids), which were diluted to 5 g/l concentration before irrigation. Each plant was treated with a different material. A reference plant was irrigated with plain water without any added material. On the day following the irrigation approximately 1 g of leaves was harvested and extracted with 50 ml of IPA/Water (4/1) for 45 min @100 C under reflux. The extract was separated from the leaves and filtered through 0.45 Nylon filter. The obtained green colored transparent liquid was tested by UV-Vis (Cary 60 uv-vis by Agilent) and fluorescence (Cary Eclipse Fluorescence Spectrophotometer by Agilent) spectrophotometers.

### Results

Extraction from Plant Treated with GQD 06-0332

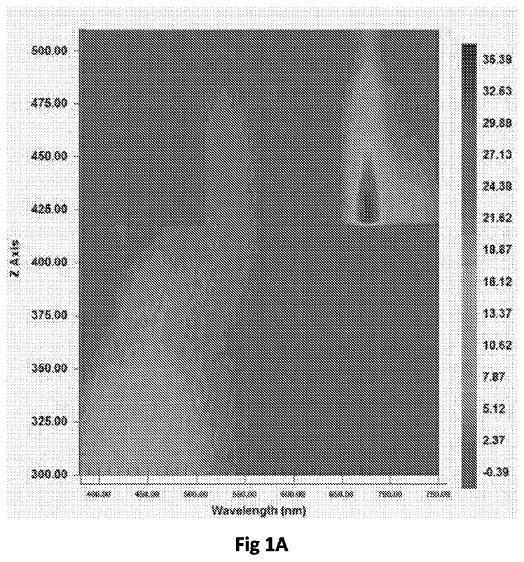

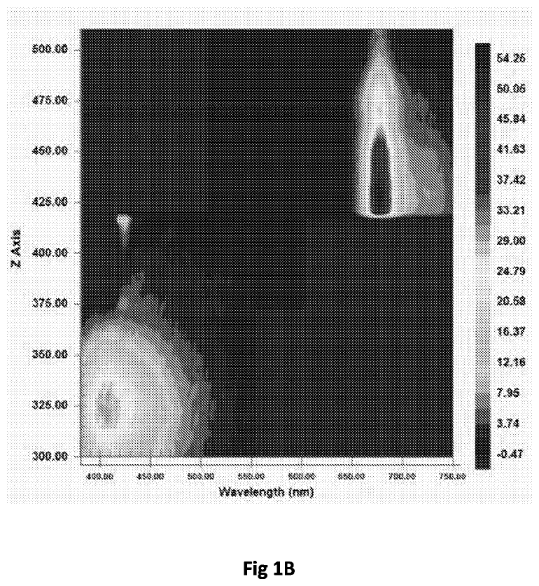

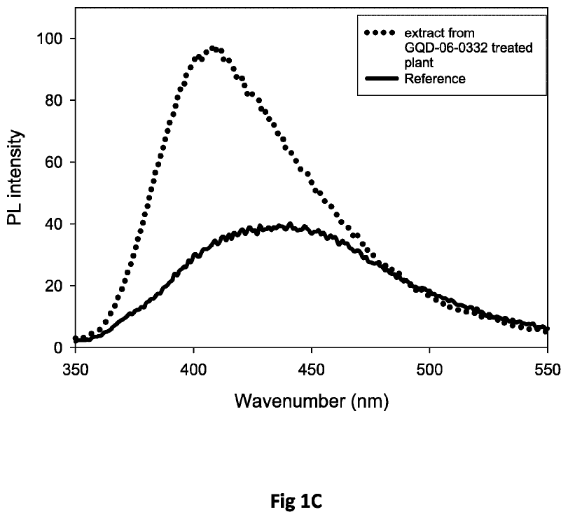

FIGS. 1 (A and B) show a 2D-Fluorescence map of extract from a reference plant (A), and extract from a plant irrigated with GQD 06-0332 (B). FIG. 1C is the spectra overlay of fluorescent peak of extract from GQD 06-0332 treated plant and the reference plant. FIG. 1 shows that extract from a plant treated with GQD 06-0332 exhibits fluorescence with maximum excitation/emission wavelength at 330/405 nm (FIGS. 1 B, C), while the reference plant extract shows weak fluorescence with maximum excitation/emission wavelength at 310/450 nm (FIGS. 1 A, C).

Extraction from Plant Treated with GQD 06-0336

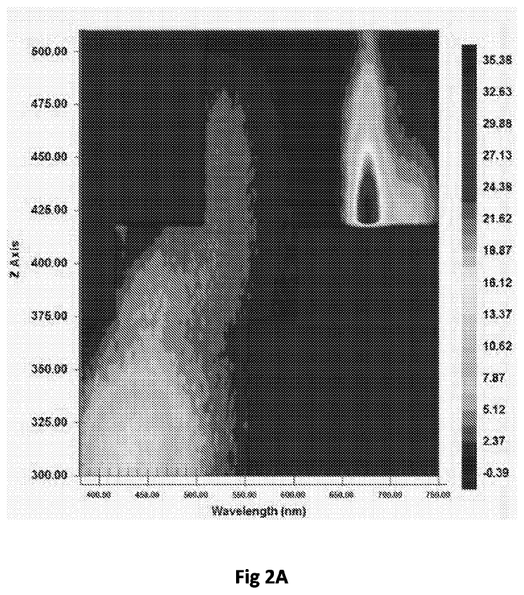

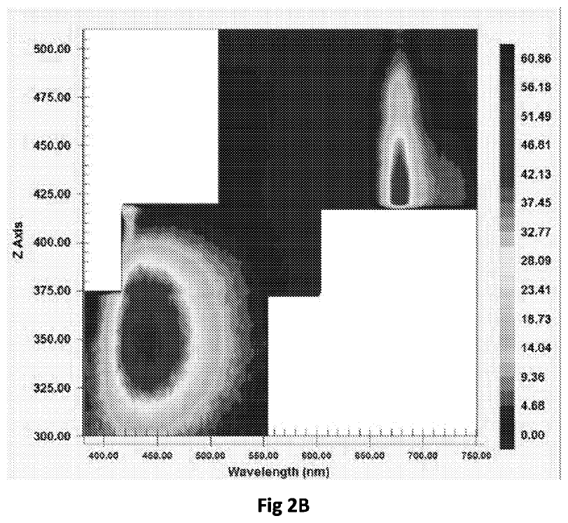

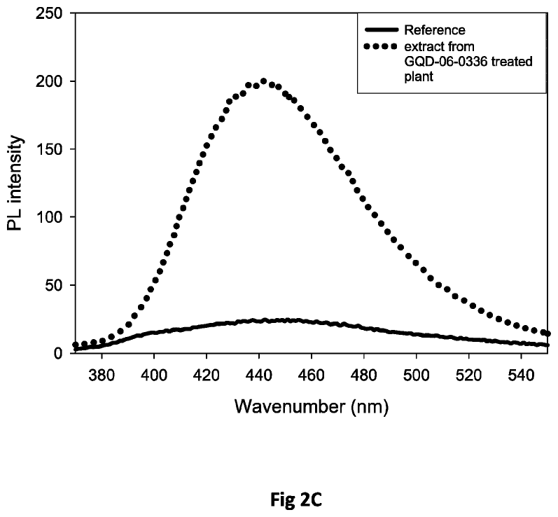

FIGS. 2 (A and B) show a 2D-Fluorescence maps of extract from reference plant (A), and extract from a plant irrigated with GQD 06-0336 (B). FIG. 2C is the PL spectra of an extract from GQD 06-0336 treated plant and from the reference plant. FIG. 2 demonstrates that the extract from a plant treated with GQD 06-0336 exhibits strong fluorescence with maximum excitation/emission wavelength at 350/450 nm (FIGS. 2 B, C), while the reference plant extract shows weak fluorescence with maximum excitation/emission wavelength at 310/450 nm (FIGS. 2 A, C).

Extraction from Plant Treated with GQD 06-0340

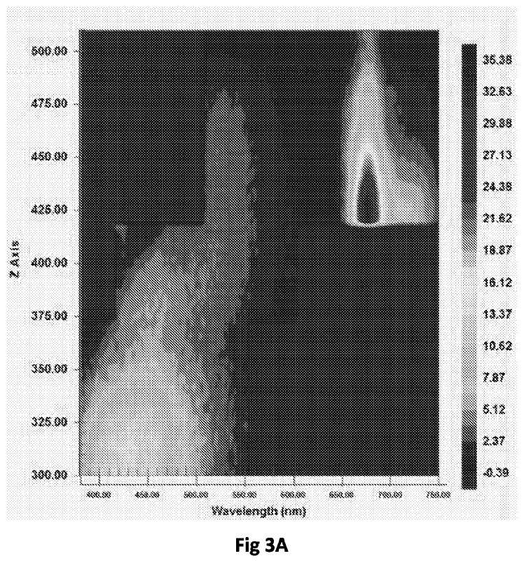

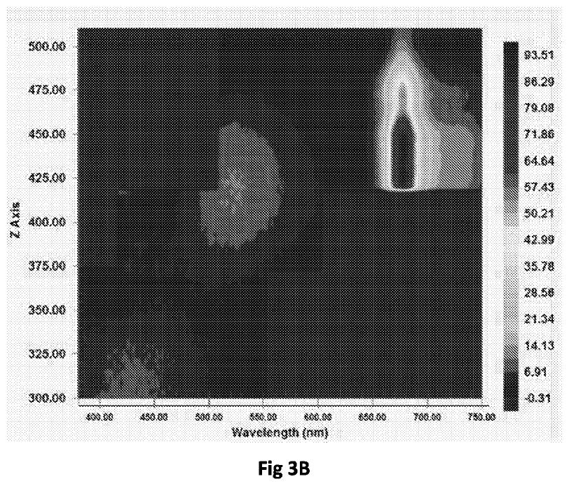

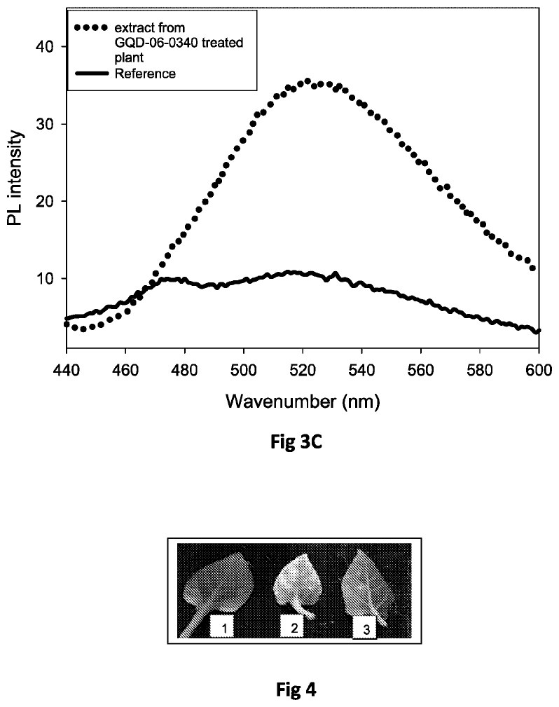

FIGS. 3 (A and B) are 2D-Fluorescence maps of ab extract from reference plant (A), and an extract from a plant irrigated with GQD 06-0340 (B). FIG. 3C is the PL spectra of extract from GQD 06-0340 treated plant and from the reference plant. This figure, like in the former two cases, illustrates the strong fluorescence effect obtained by the invention.

FIG. 4 further illustrates the invention. It is an image (taken under UV lamp) of the leaf cut from the reference plant (1), leaf from GQD 06-0332 irrigated plant (2), and leaf from GQD 06-0336 irrigated plant (3). As can be easily seen, the leaves of plants irrigated according to the invention exhibit substantial fluorescence.

### Example 2

### Extraction from Dried Plants

Three Capsicum plants were irrigated with three kinds of water-based Graphene Quantum Dots (GQDs), specifically GQD 06-0332, GQD 06-0336, and GQD 06-0340, available from STREM Chemicals, Inc., USA, (https://www.strem.com/catalog/v/96-7420/33/graphene_quantum_dots_in_water_gqds_mini_kit_liquids), which were diluted to 5 g/l concentration before irrigation. Each plant was treated with a different material. A reference plant was irrigated with plain water without any added material.

On the next day, the plants were inserted into oven with air circulation and heated @47 C for 1 h. Then, the dried leaves were extracted with 50 ml of IPA/Water (4/1) for 45 min @100 C under reflux. The extract was separated from the leaves and filtered through 0.45 Nylon filter. The obtained green colored transparent liquid was tested by UV-Vis and fluorescence spectrophotometer.

Results:

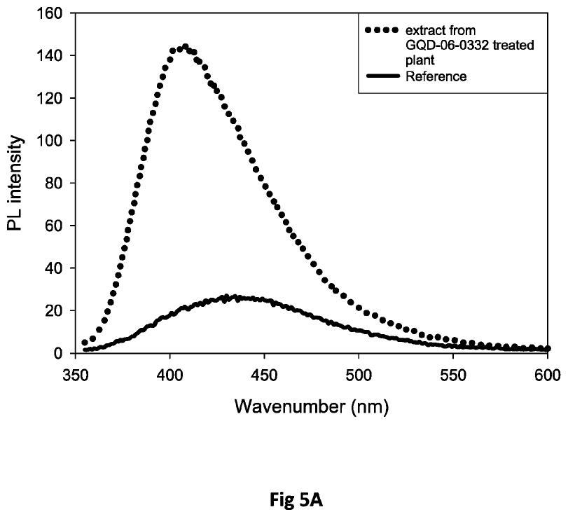

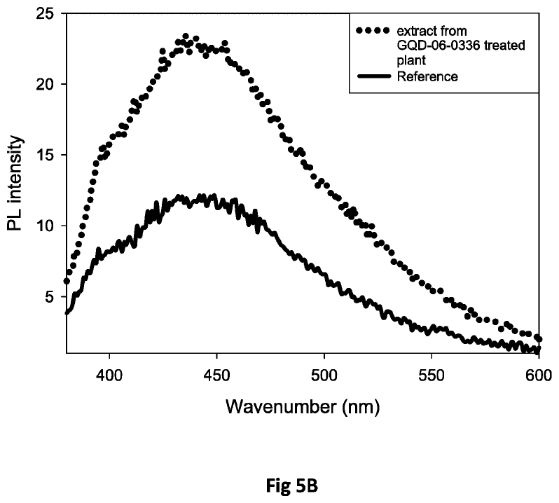

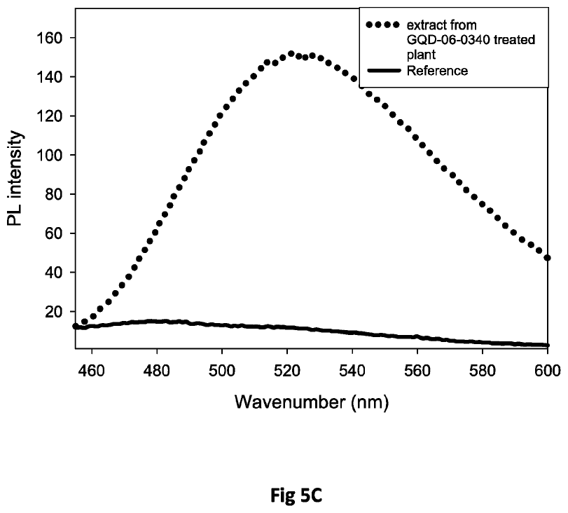

Fluorescence measurements show that GQD 06-0332, GQD 06-0336, and GQD 06-0340 can be extracted and traced from dried plants (FIGS. 5, A, C). This finding indicates that drying process does not cause damage to the tracers while they are absorbed inside the plant. On the other hand, GQD 06-0336 demonstrates low fluorescence (low concentration) after extraction from the dried plant (FIG. 5 B). It is also noticed that GQD 06-0336 gave the highest PL (compared to GQD 06-0332 and GQD 06-0340) after the extraction from freshly harvested plants. Based on these results it can be concluded that the low PL of GQD 06-0336 after drying, was probably caused by the degradation of GQD 06-0336 inside the plant during the thermal treatment.

### Example 3

Six Cannabis plants were irrigated with two kinds of water-based Graphene Quantum Dots (GQDs) (three plants with each kind of GQDs), specifically GQD 06-0336, and GQD 06-0340, available from STREM Chemicals, Inc., USA, (https://www.strem.com/catalog/v/96-7420/33/graphene_quantum_dots_in_water_gqds_mini_kit_liquids), which were diluted to 10 g/l concentration before irrigation. Each plant was treated with a different material. Three reference plants were irrigated with plain water without any added material.

On the day following the irrigation approximately 1 g of leaves was harvested and extractedwith 50 ml of IPA/Water (4/1) for 45 min @100 C under reflux. The extract was separated from the leaves and filtered through 0.45 Nylon filter. The obtained green colored transparent liquid was tested by UV-Vis (Cary 60 uv-vis by Agilent) and fluorescence (Cary Eclipse Fluorescence Spectrophotometer by Agilent) spectrophotometers.

Results:

Extraction from Cannabis Plant Treated with GQD 06-0336

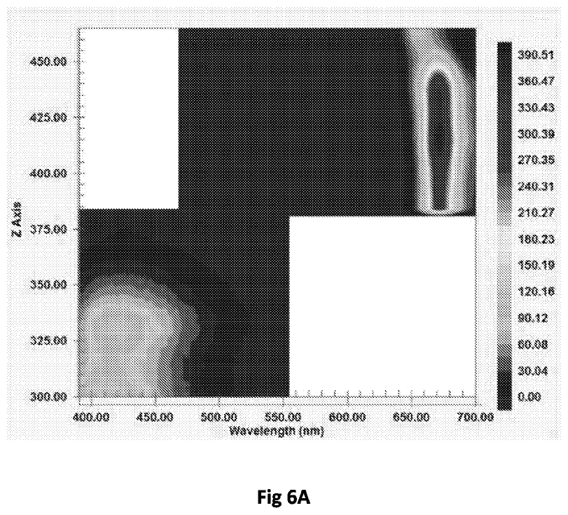

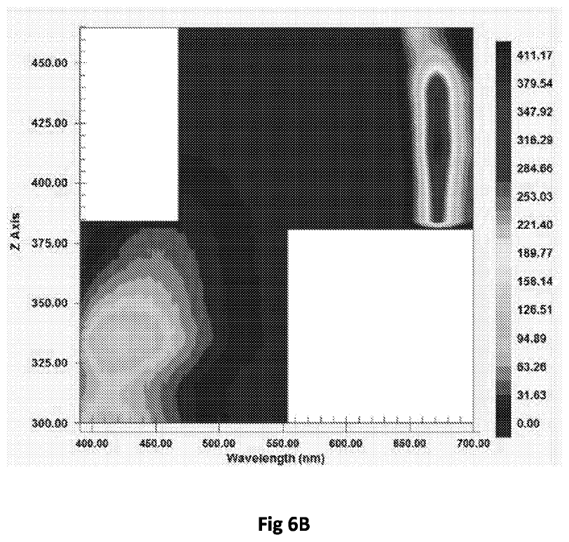

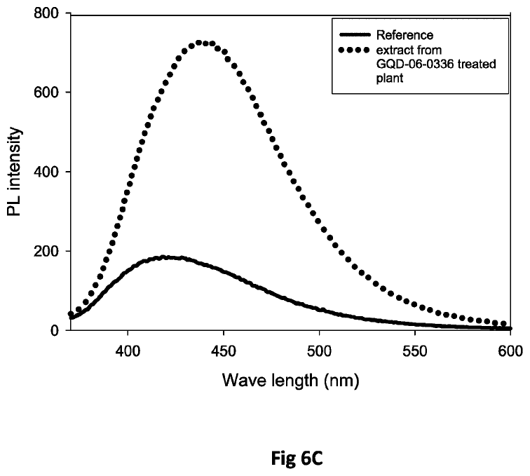

FIGS. 6 (A and B) show 2D-Fluorescence maps of an extract from reference plant (A), and of an extract from plant irrigated with GQD 06-0336 (B). FIG. 6C is the PL spectra of extract from GQD 06-0336 treated plant and from the reference plant. FIG. 2 demonstrates that the extract from a plant treated with GQD 06-0336 exhibits strong fluorescence with maximum excitation/emission wavelength at 350/450 nm (FIGS. 6 B, C), while the reference plant extract shows weak fluorescence with maximum excitation/emission wavelength at 310/450 nm (FIGS. 6 A, C).

Extraction from Cannabis Plant Treated with GQD 06-0340

FIGS. 7 (A and B) are 2D-Fluorescence maps of extract from a reference plant (A), and of an extract from a plant irrigated with GQD 06-0340 (B). FIG. 7C is the PL spectra of extract from GQD 06-0340 treated plant and from the reference plant. This figure also illustrates the strong fluorescence effect obtained this method.

### Example 4

### Stability Tests in the Marked Plants

The aim of the test is to evaluate the stability of GQDs in dried plants during storage period (up to 6 months). The cannabis plants were harvested and dried at ambient temperature. Once a month, dried leaves were extracted, and the PL of the samples was measured (according to described previously). The PL intensity was normalized to 0.1 g sample weight.

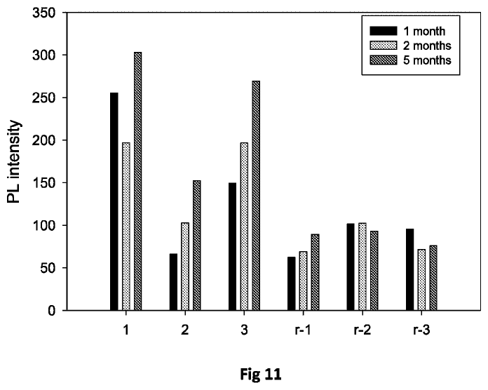

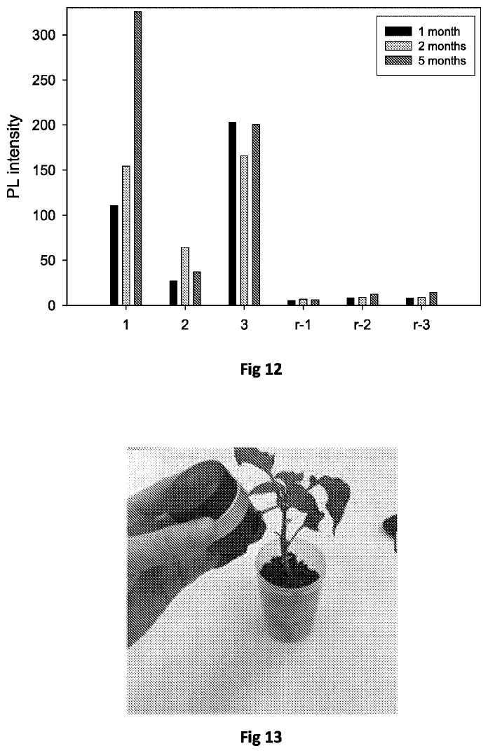

Stability tests show that during first three months of dried plants storage GQDs were not degraded, in some cases the PL even increased (FIGS. 12, 13). This phenomenon can be attributed to the continuation of the drying process during the first months of the storage.

### Example 5

### Detection of Carbon Dots in Leaves

FIG. 13 illustrates the detection of carbon dots in leaves from the plants of Example 1, marked with GQD 06-0332 and unmarked, using the InSpec™ forensic detector (manufactured by Dotz Ltd.—https://www.dotz.tech/inspec). The difference between marked and unmarked leaves is clearly seen in FIG. 4B.

Although embodiments of the invention have been described by way of illustration, it will be understood that the invention may be carried out with many variations, modifications, and adaptations, without exceeding the scope of the claims.

1. A method for tagging a plant growth, comprising incorporating one or more fluorescent carbon dots into said plant growth.

2. The method according to claim 1, wherein incorporation is performed by irrigation.

3. The method according to claim 1, wherein incorporation is performed by wetting the plant foliage.

4. The method according to claim 1, wherein the fluorescent carbon dots comprise two or more carbon dots having differently detectable maximum excitation/emission wavelengths.

5. The method according to claim 1, wherein the plant growth is selected from cannabis and Saffron.

6. A method for identifying and/or verifying the origin of plant material that was tagged with one or more carbon dots, comprising subjecting said plant material to excitation at a range of wavelengths, recording the emission obtained thereby, and comparing said emission to reference values.

7. The method according to claim 6, wherein the plant material is a live plant.

8. The method according to claim 6, wherein the plant material is a dried plant material.

9. The method according to claim 6, wherein the plant material is a plant extract.

10. A plant material tagged with one or more fluorescent carbon dots.
